SEQUENCE LISTING (32 sequences)

SEQ ID NO: 1 - protein, 133 aa, Bacillus subtilis
MSSENAQLKKDLIKAVLSPLFPTATEGGENMDSNLKALLDAAIDQKVDESETVTAESILDPSLPARWIFARITPGTTISIVTDSGDMIGPVVFVAFDQVHGIVFVTQESSVTPAGQATTLIDVDKVESVTFFS

SEQ ID NO: 2 - protein, 513 aa, Bacillus subtilis
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTETPKVGTTEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 3 - protein, 380 aa, Bacillus subtilis
MSKRRMKYHSNNEISYYNFLHSMKDKIVTVYRGGPESKKGKLTAVKSDYIALQAEKKIIYYQLEHVKSITEDTNNSTTTIETEEMLDADDFHSLIGHLINQSVQFNQGGPESKKGRLVWLGDDYAALNTNEDGVVYFNIHHIKSISKHEPDLKIEEQTPVGVLEADDLSEVFKSLTHKWVSINRGGPEAIEGILVDNADGHYTIVKNQEVLRIYPFHIKSISLGPKGSYKKEDQKNEQNQEDNNDKDSNSFISSKSYSSSKSSKRSLKSSDDQSSKSGRSSRSKSSSKSSKRSLKSSDYQSSKSGRSSRSKSSSKSSKRSLKSSDYQSSKSSKRSPRSSDYQSSRSPGYSSSIKSSGKQKEDYSYETIVRTIDYHWKRKF

SEQ ID NO: 4 - protein, 66 aa, Bacillus subtilis
MGYYKKYKEEYYTVKKTYYKKYYEYDKKDYDCDYDKKYDDYDKKYYDHDKKDYDYVVEYKKHKKHY

SEQ ID NO: 5 - protein, 195 aa, Bacillus subtilis
MGHYSHSDIEEAVKSAKKEGLKDYLYQEPHGKKRSHKKSHRTHKKSRSHKKSYCSHKKSRSHKKSFCSHKKSRSHKKSYCSHKKSRSHKKSYRSHKKSRSYKKSYRSYKKSRSYKKSCRSYKKSRSYKKSYCSHKKKSRSYKKSCRTHKKSYRSHKKYYKKPHHHCDDYKRHDDYDSKKEYWKDGNCWVVKKKYK

SEQ ID NO: 6 - protein, 357 aa, Bacillus subtilis
MCPLMAENHEVIEEGNSSELPLSAEDAKKLTELAENVLQGWDVQAEKIDVIQGNQMALVWKVHTDSGAVCLKRIHRPEKKALFSIFAQDYLAKKGMNVPGILPNKKGSLYSKHGSFLFVVYDWIEGRPFELTVKQDLEFIMKGLADFHTASVGYQPPNGVPIFTKLGRWPNHYTKRCKQMETWKLMAEAEKEDPFSQLYLQEIDGFIEDGLRIKDRLLQSTYVPWTEQLKKSPNLCHQDYGTGNTLLGENEQIWVIDLDTVSFDLPIRDLRKMIIPLLDTTGVWDDETFNVMLNAYESRAPLTEEQKQVMFIDMLFPYELYDVIREKYVRKSALPKEELESAFEYERIKANALRQLI

SEQ ID NO: 7 - protein, 82 aa, Bacillus subtilis
MKDMQPFTPVKSYTPFHSRFDPCPPIGKKYYRTPPNLYMTFQPEHMEQFSPMEALRKGTLWKDLYDFYENPYRGGDAHGKKG

SEQ ID NO: 8 - protein, 130 aa, Bacillus subtilis
MWRNASMNHSKRNDANDFDSMDEWLRQFFEDPFAWYDETLPIDLYETSQQYIIEADLTFLQPTQVTVTLSGCEFILTVKSSGQTFEKQMMLPFYFNDKNIQVECENQILTVAVNKETEDGSSFSLQFPLS

SEQ ID NO: 9 - protein, 351 aa, Bacillus subtilis
MYQKEHEEQIVSEILSYYPFHIDHVALKSNKSGRKIWEVETDHGPKLLKEAQMKPERMLFITQAHAHLQEKGLPIAPIHQTKNGGSCLGTDQVSYSLYDKVTGKEMIYYDAEQMKKVMSFAGHFHHASKGYVCTDESKKRSRLGKWHKLYRWKLQELEGNMQIAASYPDDVFSQTFLKHADKMLARGKEALRALDDSEYETWTKETLEHGGFCFQDFTLARLTEIEGEPFLKELHSITYDLPSRDLRILLNKVMVKLSVWDTDFMVALLAAYDAVYPLTEKQYEVLWIDLAFPHLFCAIGHKYYLKQKKTWSDEKYNWALQNMISVEESKDSFLDKLPELYKKIKAYREAN

SEQ ID NO: 10 - protein, 86 aa, Bacillus subtilis
MGYYKKYKEEYYTWKKTYYKKYYDNDKKHYDCDKYYDHDKKHYDYDKKYDDHDKKYYDDHDYHYEKKYYDDDDHYYDFVESYKKHH

SEQ ID NO: 11 - protein, 128 aa, Bacillus subtilis
MSFEEKVESLHPAIFEQLSSEFEQQIEVIDCENITIDTSHITAALSIQAFVTTMIIVATQLVIADEDLADAVASEILILDSSQIKKRTIIKIINSRNIKITLSADEIITFVQILLQVLNSILSELDVL

SEQ ID NO: 12 - protein, 105 aa, Bacillus subtilis
MSDNDKFKEELAKLPEVDPMTKMLVQNIFSKHGVTKDKMKKVSDEEKEMLLNLVKDLQAKSQALIENQKKKKEEAAAQEQKNTKPLSRREQLIEQIRQRRKNDNN

SEQ ID NO: 13 - protein, 172 aa, Bacillus subtilis
MESRPYSWVALDPDCDHPLDDKEKDKEKHERKCHCDVCCNGNGFFGNDNAFIDQDLAQANLNKQVSDETIIIRDSCDINVTSTDVQAVTSVVTALNAAVVTATLTSIADGVIAELVAQDLLQLTANKQVNRQKLLIECSRGVNVTTVDADIATLISTATNTLVAILVITLVL

SEQ ID NO: 14 - protein, 162 aa, Bacillus subtilis
MSCGKTHGRHENCVCDAVEKILAEQEAVEEQCPTGCYTNLLNPTIAGKDTIPFLVFDKKGGLFSTFGNVGGFVDDMQCFESIFFRVEKLCDCCATLSILRPVDVKGDTLSVCHPCDPDFFGLEKTDFCIEVDLGCFCAIQCLSPELVDRTSPHKDKKHHHNG

SEQ ID NO: 15 - protein, 408 aa, Bacillus subtilis
MNNMLDIWQSRLQEHIKETRTYMKYMLNDHLVIVLIFFLAGAASWYSKWIRDIPAHFPSFWVMAVLFSLVLTSSYVRTLLKEADLVFLLPLEAKMEPYLKQAFVYSYVSQLFPLIALSIVAMPLYFAVTPGASLVSYAAVFVQLLLLKAWNQVMEWRTTFQNDRSMKRMDVIIRFAANTLVLYFVFQSVYMYALLVYVIMAVLYLYMSSAAKRKTFKWESHIESELRRKQRFYRIANLFTDVPHLRKQAKRRAYLDFLLRLVPFEQRKTFAYMFTRAFLRSSDYLGILVRLTIVFALIIMYVSASPLIAAVLTVFAIFITGIQLLPLFGHFDHLALQELYPVQKETKLKSYFSLLKTALSIQALLMSVASAYAAGLTGFLYALIGSAVLIFVVLPAYMTTRLKKHGKL

SEQ ID NO: 16 - protein, 392 aa, Bacillus subtilis
MLLEQQPINHEDRNVPQPIRSDGAGAIDTGPRNIIRDIQNPNIFVPPVTDEGMIPNLRFSFSDAPMKLDHGGWSREITVRQLPISTAIAGVNMSLTAGGVRELHWHKQAEWAYMLLGRARITAVDQDGRNFIADVGPGDLWYFPAGIPHSIQGLEHCEFLLVFDDGNFSEFSTLTISDWLAHTPKDVLSANFGVPENAFNSLPSEQVYIYQGNVPGSVASEDIQSPYGKVPMTFKHELLNQPPIQMPGGSVRIVDSSNFPISKTIAAALVQIEPGAMRELHWHPNSDEWQYYLTGQGRMTVFIGNGTARTFDYRAGDVGYVPSNAGHYIQNTGTETLWFLEMFKSNRYADVSLNQWMALTPKELVQSNLNAGSVMLDSLRKKKVPVVKYPGT

SEQ ID NO: 17 - protein, 254 aa, Bacillus subtilis
MNHFYVWHIKRVKQLIIILIAAFAAASFFYIQRAVPLPVFSTDTGPKAIYKGETDSKDISLTFDISWGDERAEPILNTLKANGIKNATFFLSASWAERHPDTVARIVKDGHQIGSMGYAYKNYANLESSEIKKDMNRAQTAFEKLGVKDIQLLRPPTGQFNKNVLKVAKQYNYTVVHYSVNSQDWTNPGVEKIIDNVTKQVSGGDIILLHASDSAKQTEEALPDIIHQLKEKGLKNVTVGDLIANSDAKSAEVK

SEQ ID NO: 18 - protein, 290 aa, Bacillus subtilis
MQFQIGDMVARKSYQMDVLFRIIGIEQTSKGNSIAILHGDEVRLIADSDFSDLVAVKKDEQMMRKKKDESRMNESLELLRQDYKLLREKQEYYATSQYQHQEHYFHMPGKVLHLDGDEAYLKKCLNVYKKIGVPVYGIHCHEKKMSASIEVLLDKYRPDILVITGHDAYSKQKGGIDDLNAYRHSKHFVETVQTARKKIPHLDQLVIFAGACQSHFESLIRAGANFASSPSRVNIHALDPVYIVAKISFTPFMERINVWEVLRNTLTREKGLGGIETRGVLRIGMPYKSN

SEQ ID NO: 19 - protein, 453 aa, Bacillus subtilis
MNPKRFLIGIDKTSENTLFLPSSLKQDGLLHAAFGTKVVRCHVAYRRHLEQTVLLSENLFHELLLPHRSRADILIHDHTVHIGPLVGIFTAGFTVSLERPFKDRSLFFSKLVTLHEQAGGYCFVFGAHQINWEEGTIEGLLYRENGWEKKIVPLPNVVYDRLPNRKIEDSLLLQHTKKRLIDEYQIPWFNKTFFNKWNVHQLLEKDPRTAPFLPRSELTPSVELIDELCGAYKKVYIKPANGALGTGIYQLTRTDGGLTVKHTNDAKTFTSIDYSDAASFLAEFQKHHNPSDFLIQQGVDLIEFQGKPADFRVHTNKNRKGKWTVTAIAVKISGKNSITTHLSNGGTVKTLAEVYDDPAERVEVIKKLSAAALTASHVLHDHIEGFIGEIGFDFGIDQNGKVWMFEANSRPGRSIFSHPNLHHVDSLTKRRSFEYASYLSEKAITSPEALWPS

SEQ ID NO: 20 - protein, 28 aa, Bacillus subtilis
MSGGYSNGFALLVVLFILLIIVGAAYIY

SEQ ID NO: 21 - protein, 288 aa, Bacillus subtilis
MSKQLLALNIDGALLRSNGKIHQATKDAIEYVKKKGIYVTLVTNRHFRSAQKIAKSLKLDAKLITHSGAYIAEKIDAPFFEKRISDDHTFNIVQVLESYQCNIRLLHEKYSIGNKKKVNSNLLGKALIHPSDPIFYPVQFVESLSDLLMDEPVSAPVIEVYTEHDIQHDITETITKAFPAVDVIRVNDEKLNIVPKGVSKEAGLALVASELGLSMDDVVAIGHQYDDLPMIELAGLGVAMGNAVPEIKRKADWVTRSNDEQGVAYMMKEYFRMQQRKGFLDKFHMKRV

SEQ ID NO: 22 - protein, 145 aa, Bacillus subtilis
MHYSYYPYPVPYREDPVLIRNLQKAINGEFSAIQCYRKLAELAHSDEVRKQIEEIRRDEIRHLREFSTLYGSITGKHIMPKQTEECPDNFTRGLDAAFKDEQETVDFYLRAAEETSNLKAKGVFTRAARDEQNHAVWFLYYLMER

SEQ ID NO: 23 - protein, 247 aa, Bacillus subtilis
MFSPQQPMRNYPQPGPPRPMPNQRMMGRRPPNMRGPSFGAQQQGFQQAGQFLPQANAGARNGAGAGGGLKGMLSRFLPGGGGAGSAGVPGIPGAGAAASGGAGLQGIQNIANPASLSSMLGNVQKVLGMAQQVTPMIQQYGPLVRNLPAMMKLYSQLSKSDDTETEANEESEKQSVSEESSEKEKETETKTSDGNKKSKPKSSSLPKKSKTTDNKEQELKTSAPKKKEAASPAPKRTSGSSKPRLYI

SEQ ID NO: 24 - protein, 186 aa, Bacillus subtilis
MYYNPFSPQCYYYVTVPMYNDGRSVYWTIPNEMEKVHRGADLRSSYEDRNLLLKDYGPKPFVVNINRATKQNNTFRTALWTGKHFQVTLMSLGIGEDIGLEIHPNVDQFLRIEQGRGIVKMGKSKDHLNFQRNVYDDSAIVVPAGTWHNVINTGNTPLKLYSIYAPPNHPFGTVHETKADAVAAED

SEQ ID NO: 25 - protein, 407 aa, Bacillus subtilis
MKLAFICTEKLPAPAVRGGAIQMMIDGVTPYFSSRYDLTIFSIEDPSLPKRETKDGVHYIHLPKEHYREAVAEELRASSFDLIHVFNRPLNVSLYKKASPNSKIVLSLHNEMFSEKKMTFAQGKEVLDNVSMITTVSEFIKQTVIERFPEAEDITKVVYSGVDLNSYPPVWTMKGSAVRKTYRKKYGIEDKKVILFAGRLSPTKGPHLLIHSMRRILQQHPDAVLVIAGGKWFSDDSENQYVTYLRTLALPYRDHIIFTKFIPADDIPNLFLMADVFVCSSQWNEPLARVNYEAMAAGTPLITTNRGGNGEVVKHEVTGLVIDSYNKPSSFAKAIDRAFTDQELMNKMTKNARKHVEALFTFTHAAKRLNTVYQSVLTPKNKQFPPPFLTQNFDLSSINQLFVKAKT

SEQ ID NO: 26 - protein, 113 aa, Bacillus subtilis
MDQEAGFMVNFINSYFIALGVLIGGALIGGLGAYLAGEPPLTAITKLANRLKIWALVAAIGGTFDAVYSFERGILEGNTRDIFKQLLLIISAMGGAQSGWLIISWLTQEHLSS

SEQ ID NO: 27 - protein, 13 aa, synthetic construct
AHIVMVDAYKPTK

SEQ ID NO: 28 - protein, 139 aa, synthetic construct
MSYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 29 - protein, 14 aa, synthetic construct
VPTIVMVDAYKRYK

SEQ ID NO: 30 - protein, 144 aa, synthetic construct
MSYYHHHHHHDYDIPTTENLYFQGAMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 31 - protein, 16 aa, synthetic construct
RGVPHIVMVDAYKRYK

SEQ ID NO: 32 - protein, 143 aa, synthetic construct
SYYHHHHHHDYDIPTTENLYFQGAMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGS

(19) United States
(12) Patent Application Publication
(10) Pub. No.: US 2026/0286282 A1
(43) Pub. Date: Sep. 24, 2026

(54) METHODS FOR SPORE DISPLAY

(71) Applicant: Trustees of Tufts College, Medford, MA (US)

(72) Inventors: Trevor Nicks, Medford, MA (US); Todd Chappell, Medford, MA (US); Nikhil Nair, Medford, MA (US)

(21) Appl. No.: 18/874,058

(22) PCT Filed: Jun. 13, 2023
(86) PCT No.: PCT/US2023/068379

§ 371 Date: Dec. 11, 2024

Related U.S. Application Data
(60) Provisional application No. 63/351,606, filed on Jun. 13, 2022.

(51) Int. Cl. C12N 1/20; A61K 38/43; A61K 47/42; C07K 1/14; C07K 14/32; C12R 1/125

(52) U.S. Cl. CPC C12N 1/20; A61K 38/43; A61K 47/42; C07K 1/14; C07K 14/32; C07K 2319/70; C12R 2001/125

(57) ABSTRACT

The present invention provides engineered bacterial spores that comprise a cargo protein conjugated to a spore coat protein. Also provided are bacteria and methods for producing the spores and methods of using the spores to deliver a cargo protein to a subject. Spores are a highly stable phenotype of spore-forming microbes like Bacillus subtilis. Spores have robust tolerance to thermal, pH, proteinase, and solvent challenges and can be used as protein carriers for many potential applications, including vaccines, probiotic delivery, bioremediation, enzyme immobilization, bioprocessing, and more. Spore display is a technique for loading proteins of interest within a spore or onto the surface of a spore. This is accomplished by conjugating a protein of interest to a spore coat protein to anchor it to the spore.

### CROSS-REFERENCE TO RELATED APPLICATIONS

This application claims priority to U.S. Provisional Application No. 63/351,606 filed on Jun. 13, 2022, the contents of which are incorporated by reference in their entireties.

### SEQUENCE LISTING

This application includes a sequence listing in XML format titled “166118.01328_ST26.xml”, which is 34,921 bytes in size and was created on Jun. 13, 2023. The sequence listing is electronically submitted with this application via Patent Center and is incorporated herein by reference in its entirety.

### BACKGROUND

Spores are a highly stable phenotype of spore-forming microbes like Bacillus subtilis. Spores have robust tolerance to thermal, pH, proteinase, and solvent challenges and can be used as protein carriers for many potential applications, including vaccines, probiotic delivery, bioremediation, enzyme immobilization, bioprocessing, and more. Spore display is a technique for loading proteins of interest within a spore or onto the surface of a spore. This is accomplished by conjugating a protein of interest to a spore coat protein to anchor it to the spore. There are at least 44 B. subtilis spore coat proteins that could potentially function as anchor proteins for spore display, but only 12 of these proteins have previously been used to display enzymes. Thus, to improve the modularity of B. subtilis spore display methods, additional anchor proteins and display strategies must be identified.

### SUMMARY

In a first aspect, the present disclosure provides bacterial spores comprising a cargo protein conjugated to a spore coat protein.

In a second aspect, the disclosure provides bacteria that produce the spores described herein.

In a third aspect, the disclosure provides methods for producing the spores described herein. In some embodiments, the methods comprise culturing a bacterium that expresses a fusion protein comprising the spore coat protein and the cargo protein in a sporulation medium.

In other embodiments, the methods comprise culturing a bacterium that expresses both (i) the spore coat protein fused to a first protein tag and (ii) the cargo protein fused to a second protein tag that specifically binds to the first protein tag in a sporulation medium.

In further embodiments, the methods comprise (a) culturing a first bacterium that expresses the spore coat protein fused to a first protein tag in a sporulation medium; (b) culturing a second bacterium that expresses the cargo protein fused to a second protein tag that specifically binds to the first protein tag; (c) isolating the cargo protein produced by the second bacterium in step b; and (d) combining with the spores produced by the first bacterium in step a with the cargo protein isolated from the second bacterium in step c.

In a fourth aspect, the disclosure provides pharmaceutical compositions comprising a pharmaceutically acceptable carrier and an engineered spore described herein.

In a fifth aspect, the disclosure provides methods for delivering a cargo protein to a subject in need thereof. The methods comprise administering a pharmaceutical composition described herein to the subject.

### BRIEF DESCRIPTION OF THE DRAWINGS

FIG. 1 is a schematic depicting the co-transformation assay described in Example 1. In this assay, Bacillus subtilis is transformed with two DNA molecules with orthogonal integration sites. The primary marker (kanR) provided on one DNA molecule enables selection for single colonies. The secondary marker (GusA) provided on the other DNA molecule enables identification of functional spore coat protein (SCP)-GusA fusion proteins.

FIG. 2 is a graph showing the activity of the enzyme GusA when expressed from 34 different constructs as a fusion protein with 26 different SPC anchors. Values are the average of three biological replicates. Standard error is shown with error bars. “WT” indicates wildtype spores of B. subtilis.

FIG. 3 is a graph showing the surface availability of the enzyme GusA when expressed from 34 different constructs as a fusion protein with 26 different SPC anchors. The median and upper and lower quartile ranges of fluorescence values for recombinant spores labeled with an anti-GusA polyclonal antibody are shown.

FIG. 4 is a schematic depicting a method for cis-loading a cargo protein (i.e., an enzyme) onto the B. subtilis spore surface via direct fusion to a spore coat protein.

FIG. 5 is a schematic depicting a method for cis-loading a cargo protein onto the B. subtilis spore surface via indirect conjugation using the SpyCatcher-SpyTag system.

FIG. 6 is a schematic depicting a method for trans-loading a cargo protein onto the B. subtilis spore surface via indirect conjugation using the SpyCatcher-SpyTag system.

FIG. 7 is a graph showing the relative activity of the enzyme GusA at various temperatures as a free enzyme and when fused to the C-terminus of the anchor proteins CgeA, CotG, CotX, CotY, and SscA. Spore-displayed GusA was incubated at 25, 37, 50, and 60° C. Values are the average of three biological replicates.

FIGS. 8A-8C are a series of graphs showing the relative activity of the enzyme GusA at various pH levels, both as a free enzyme (FIG. 8A) and when it is fused to the anchor proteins CotX (FIG. 8B) and SscA (FIG. 8C). Buffers use are 50 mM citrate (C), phosphate (P), and Tris (T).

FIG. 9 is a graph showing the activity of the enzyme esterase when expressed as a free enzyme and when fused to C-terminus of the anchor proteins CgeA, CotG, CotX, CotY, and SscA. Values are the average of two biological replicates.

### DETAILED DESCRIPTION

The present invention provides engineered bacterial spores that comprise a cargo protein conjugated to a spore coat protein. Also provided are bacteria and methods for producing the spores and methods of using the spores to deliver a cargo protein to a subject.

Bacterial spores have robust tolerance to thermal, pH, proteinase, and other stability challenges and can thus be used as protein carriers in various applications. The Bacillus subtilis spore coat comprises more than 44 known spore coat proteins, all of which could potentially serve as anchor proteins for spore display. However, only 12 of these spore coat proteins have ever been used to display enzymes. Thus, to identify novel anchor proteins, the inventors tested the ability of the 44 known B. subtilis spore coat proteins to be used to display the enzyme beta-glucuronidase (GusA). From this screen, they identified 13 novel spore coat proteins that can serve as anchor proteins for enzymes in addition to the 12 that were previously used.

The newly identified anchor proteins may each offer different advantages for use in specific applications. For example, some of these anchor proteins may be better suited for facilitating catalysis because they are great for stabilizing enzymes, whereas others may be better suited for use as vaccines because they offer increased antigen surface availability. Thus, by increasing the number of known B. subtilis anchor proteins, the inventors have expanded the toolbox of available proteins for use in spore display and have increased the likelihood that a suitable anchor protein will be available for a particular application. The inventors have tested the ability of a subset of the identified B. subtilis anchor proteins to stabilize conjugated enzymes against harsh conditions (i.e., high temperature and extreme pH). Their data suggests that the spore coat protein SscA is an excellent display anchors that is as good as, if not better than, commonly used anchor proteins (e.g., CotB, CotC, CotG, CotX, CotY, and CotZ).

Traditional spore display methods rely on direct fusion of a cargo protein to a spore coat protein. For these methods to work, the resulting fusion protein must be properly expressed, folded, and loaded onto the spore during sporulation. However, fusion proteins are often misfolded and even a properly folded fusion protein can interfere with spore assembly, preventing loading of the cargo protein and/or weakening the spore structure. These issues become more serious as protein size increases. Thus, these methods may not work when the cargo protein is a large protein such as a multimeric enzyme or a large antibody.

To circumvent this issue, the inventors have designed a spore display system that does not rely on the direct fusion of the cargo protein to an anchor protein. Instead, their system uses pairs of protein tags that spontaneously form a covalent bond (e.g., SpyCatcher/SpyTag), allowing the cargo protein to fold independently from the anchor protein before it is loaded onto the spore. Their cargo loading system is advantageous because (1) it will allow many more proteins to be functionally loaded into or onto spores, and (2) it will decrease the length of design-build-test cycles for spore display.

B. subtilis spore display is useful for a variety of applications, including as a means for delivering therapeutic proteins and as an enzyme immobilization strategy for use in synthetic biochemistry platforms. These applications have traditionally relied on the use of a recombinant production organism (e.g., Escherichia coli). However, in some applications, there is risk associated with the escape of recombinant genetic material into the environment. Recombinant genetic material can provide new functions to organisms, which could have unintended and potentially disastrous consequences (e.g., the spread of antibiotic resistance genes). Proteins are inherently “agenetic,” meaning that they do not carry the genetic material necessary for an organism to make copies of them. Thus, for increased safety, proteins can be used after they have been purified away from the genetic material that encodes them (e.g., in a cell-free system). However, in such methods, protein instability leads to challenges for both protein storage and delivery. Spore display is known to confer stability to covalently loaded proteins, increasing their storage lifetime and their robustness in the face of stability challenges (e.g., pH extremes, temperature swings, solvents, and detergents). Spore display also simplifies the protein purification process because spores can be easily isolated and concentrated by centrifugation and/or filtration. Further, agenetic spores (i.e., spores that deliver a cargo protein in the absence of genetic material encoding that protein) can be made by trans-loading cargo proteins produced by another cell onto the spore surface, eliminating the risk associated with the escape of potentially harmful recombinant genetic material.

### Spores:

In a first aspect, the present invention provides bacterial spores comprising a cargo protein conjugated to a spore coat protein.

A “bacterial spore” or “spore” is a stripped-down, metabolically inactive form into which certain bacteria in the phylum Bacillota can reduce themselves. When spores are formed intracellularly, they are sometimes referred to as “endospores”. Spores enable bacteria to lie dormant for extended periods, even centuries, as they are very resistant to harsh conditions (e.g., high temperatures, radiation, desiccation, and chemical agents). Spore formation is usually triggered by a lack of nutrients. As a bacterium begins the process of forming a spore, it divides asymmetrically forming a larger mother cell and a smaller forespore. The forespore is engulfed by the mother cell, and then undergoes dehydration and maturation into a spore. Finally, the mother cell is destroyed via programmed cell death and the spore is released into the environment. When the environment becomes more favorable, the spore can reactivate itself into a vegetative state.

The engineered spores of the present invention are designed to be used for spore display. “Spore display” is a technique for loading proteins of interest within a spore or onto the surface of a spore. Spore display can be used to stabilize and/or deliver a cargo protein. As used herein, the term “cargo protein” refers to a protein that is conjugated to a spore. In some embodiments, the cargo protein is an enzyme, pharmaceutical, antibody, small-molecule, or another genetically encoded molecule. As used herein, an “enzyme” is a protein that functions as a catalyst (i.e., speeds up the rate of a specific biochemical reaction). Examples of suitable enzymes for use as cargo proteins include, without limitation, cellulases (e.g., cellulase from Clostridium cellulovorans), dehydrogenases (e.g., xylose reductase from Neurospora crassa, phosphite dehydrogenase from Pseudomonas stutzeri, formate dehydrogenase, formaldehyde dehydrogenase, alcohol dehydrogenase, acetaldehyde dehydrogenase, aldehyde dehydrogenase), agarases (e.g., agarase from Pseudomonas vesicularis, agarase from Zobellia galactanivorans), lyases (e.g., phenylalanine ammonia lyase from Anabaena variabilis), isomerases (e.g., L-arabinose isomerase from Lactobcillus sakei), antibiotic-degrading enzymes (e.g., beta-lactamase from Escherichia coli, extended-spectrum beta-lactamases, macrolide esterase, N-acetyltransferase, O-adenyltransferase, chloramphenicol acetyltransferase, AAC (6′)-Ib-cr acetyltransferase), phosphorylases (e.g., trehalose phosphorylase from Thermoanaerobacter brockii, sucrose phosphorylase from Bifidibacterium adoloscentis) decarboxylases (e.g., malonate decarboxylase from Geobacillus stearothermophilus and its subunits), and plastic-degrading enzymes (e.g., PETase). Other suitable enzymes are also contemplated.

The cargo protein is attached to the spore coat of the spore via conjugation to a spore coat protein. The “spore coat” is a proteinaceous shell that encapsulates the genomic material of the bacterial spore and plays a major role in its survival. As used herein, the term “spore coat protein” refers to any protein that can form part of a spore coat. As is described in Example 1, the inventors tested 44 known B. subtilis spore coat proteins for the ability to serve as anchor proteins for spore display. As used herein, an “anchor protein” or a “spore coat anchor protein” is a spore coat protein to which a cargo protein can be conjugated without disrupting its ability to be loaded into a spore. From their screen, the inventors identified 26 functional anchor proteins, of which 13 were novel (i.e., had not previously been shown to function as anchor proteins). Thus, in some embodiments, the spore coat protein is selected from the list of 26 functional anchor proteins provided in Table 2. In preferred embodiments, the spore coat protein is selected from the list of 13 novel functional anchor proteins provided in Table 3. In specific embodiments, the spore coat protein is SscA.

The cargo may be conjugated to the spore coat protein by any means known in the art. As is described in Example 3, cargo proteins may be conjugated either directly or indirectly to a spore coat protein. For example, in some embodiments, the spore coat protein and the cargo protein are conjugated directly via expression of these proteins as a fusion protein. A “fusion protein” is a protein comprising at least two domains that are each encoded by separate genes that have been joined such that they are transcribed and translated as a single unit, producing a single polypeptide. The spore coat protein and the cargo protein may be fused together in any order, i.e., either the C-terminus of the spore coat protein can be fused to the N-terminus of the cargo protein, or the N-terminus of the spore coat protein can be fused to the C-terminus of the cargo protein. The inventors determined that, for the enzyme GusA, fusing the N-terminus of the enzyme to the C-terminus of the spore coat protein is preferable (i.e., best maintains enzyme function). However, different orientations will be preferable for other enzymes.

Unfortunately, fusion proteins are often misfolded and even a properly folded fusion protein can interfere with spore assembly. Thus, in other embodiments, the spore coat protein and the cargo protein are conjugated indirectly via fusion of each component to a different member of a protein tag pair. A “protein tag” is a short amino acid sequence that is added to another protein. Many protein tags are known in the art and include, for example, myc tags, FLAG tags, hemagglutinin tags, polyhistidine tags, and strep tags. Advantageously, due to the small size of these tags (typically 5-15 amino acids), the addition of a protein tag generally has no effect on the function of the protein to which it is attached. As used herein, a “protein tag pair” refers to two protein tags that bind to each other specifically. The term “specific” refers to the ability of a protein to bind one molecule in preference to other molecules. Under appropriate conditions, a protein that specifically binds to a target molecule will bind to that target molecule without binding to other molecules present in a sample in a significant amount. Specific binding can mean binding to a target molecule with an affinity that is at least 25% greater, at least 50% greater, at least 100% (2-fold) greater, at least ten times greater, at least 20-times greater, or at least 100-times greater than the affinity to any other molecule.

One major problem with most of the protein tags commonly used in the art is the instability of their interactions with their non-covalent binding partners. Stable, covalent conjugation of the spore coat protein and the cargo protein is preferable to non-covalent conjugation, as it would help to be able to keep cargo proteins properly displayed on the spore surface as the spores encounter harsh environments. Covalent conjugation would also be preferable for applications in which the functionalized spores are used as long-term enzyme storage devices, therapeutic delivery vehicles, or catalytic microparticles. Thus, in preferred embodiments, the protein tag pair forms a covalent bond. A “covalent bond” is a strong chemical bond that arises when two atoms share a pair of electrons.

In some embodiments, the covalent bond is an isopeptide bond. An “isopeptide bond” is an amide bond formed between a carboxyl/carboxamide group of one amino acid and an amino group of another amino acid, wherein at least one of these groups is outside of the protein backbone. Isopeptide bonds are stable under conditions in which non-covalent interactions would rapidly dissociate, e.g., over long periods of time (e.g., weeks), at high temperature (>95° C.), under high force, or with harsh chemical treatment (e.g., pH 2-11, organic solvents, detergents, denaturants). Isopeptide bonds are irreversible under biological conditions and are resistant to most proteases. Examples of protein tag pairs that form an isopeptide bond include, without limitation, SpyTag/SpyCatcher, SpyTag002/SpyCatcher002, SpyTag003/SpyCatcher003, SnoopTag/SnoopCatcher, DogTag/DogCatcher, and SdyTag/SdyCatcher. Isopeptide bonds can also be formed via sortase-mediated ligation, butelase-mediated ligation, disulfide bonds formation, and using non-canonical amino acids.

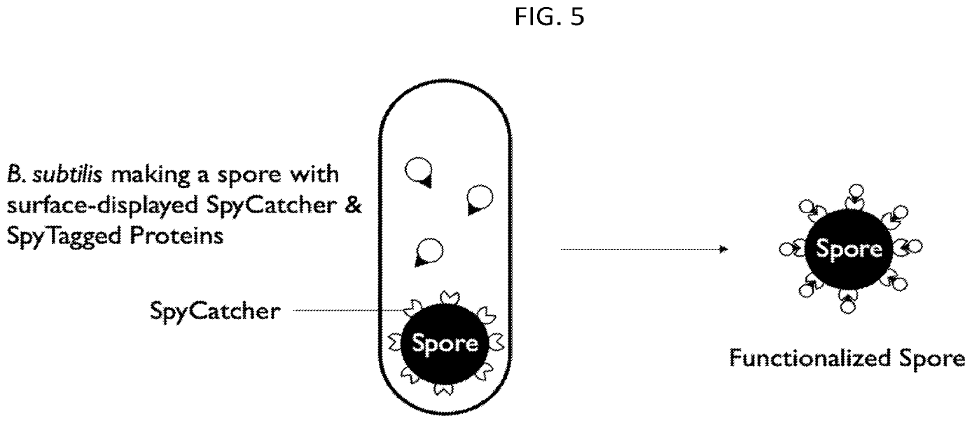

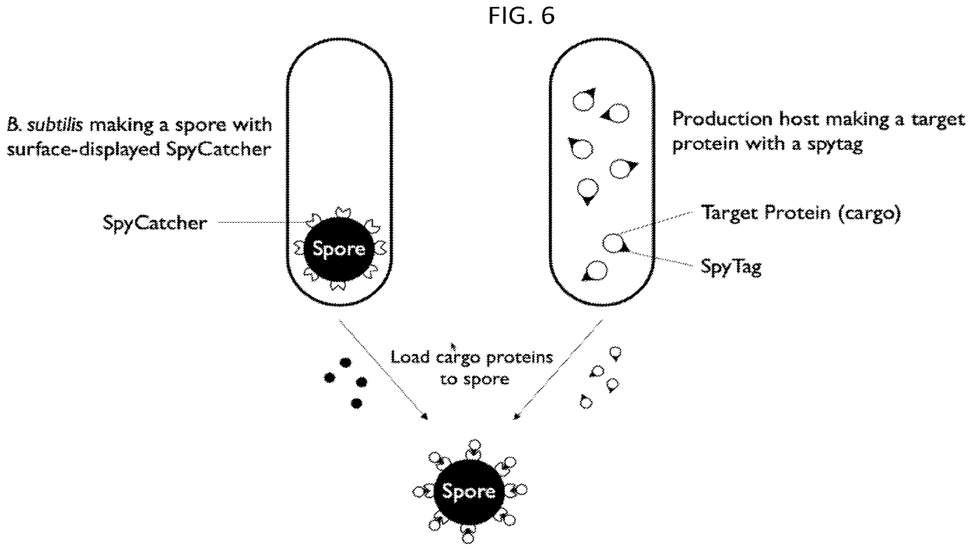

In Example 3, the inventors describe two methods by which a cargo protein can be conjugated to a spore coat protein using the SpyCatcher-SpyTag system (see FIG. 5 and FIG. 6). Specifically, the inventors have designed a method in which the spore coat protein is expressed as a fusion protein with SpyCatcher and the cargo protein is expressed as a fusion protein with SpyTag. When these protein tags spontaneously form an isopeptide bond, they indirectly conjugate the cargo protein to the spore coat protein. Thus, in some embodiments, the first protein tag is SpyCatcher and the second protein tag is a SpyTag. In other embodiments, the first protein tag is SpyTag and the second protein tag is SpyCatcher, such that the spore coat protein is fused to SpyTag and the cargo protein is fused to SpyCatcher.

The SpyCatcher-SpyTag system is described in detail in U.S. Pat. No. 9,547,003, which is hereby incorporated by reference in its entirety. In this system, the peptide Spy Tag (SEQ ID NO: 27; 13 amino acids) spontaneously reacts with the protein SpyCatcher (SEQ ID NO: 28; 12.3 kDa) to form an isopeptide bond. SpyTag and SpyCatcher were formed by splitting and engineering the CnaB2 domain of the protein FbaB from Streptococcus pyogenes, which naturally forms an intramolecular isopeptide bond. Specifically, SpyTag was formed from the C-terminal beta strand of CnaB2, which contains the reactive aspartic acid D556, and SpyCatcher was formed from the rest of the beta strands, which contain the reactive lysine K470 and the catalytic glutamic acid E516. DNA sequences encoding SpyTag and SpyCatcher can be recombinantly introduced into DNA sequence encoding two proteins of interest to form two fusion proteins (i.e., one comprising SpyTag and one comprising SpyCatcher). The two fusion proteins can then be covalently linked by simply mixing them in a reaction. Suitable conditions for this reaction are known in the art. For example, this reaction may be performed in vitro in phosphate buffer saline (PBS) or in vivo in the cytoplasm of the cell.

Subsequent generations of the SpyCatcher-SpyTag system offer faster binding than the original. For example, in the second generation, SpyTag002 (SEQ ID NO: 29)/SpyCatcher002 (SEQ ID NO: 30), which was created through phage display, the protein tag pair reacts up to 12 times faster than the original pair (rate constant: 2.0±0.2×10⁴ M⁻¹ s⁻¹). In the third generation, SpyTag003 (SEQ ID NO: 31)/SpyCatcher003 (SEQ ID NO: 32), which was created through rational design, the protein tag pair reacts up to 400 times faster than the original pair (rate constant: 5.5±0.6×10⁵ M⁻¹ s⁻¹). Thus, in some embodiments, the protein tag pair used to conjugate the cargo protein to the spore coat protein is SpyTag002/SpyCatcher002 or SpyTag003/SpyCatcher003.

### Bacteria:

In a second aspect, the present invention provides engineered bacteria that produce the cargo-loaded spores described herein.

As is described in Example 3, the cargo-loaded spores of the present invention can be produced using either direct protein fusion or indirect protein conjugation. Thus, the bacteria of the present invention may be genetically engineered (1) to express a single fusion protein comprising the cargo protein fused to the spore coat protein, or (2) to express the spore coat protein and the cargo protein as individual proteins (i.e., as proteins that are separately transcribed and translated). In embodiments in which the bacterium expresses the spore coat protein and the cargo protein as individual proteins, protein tag pairs can be used to indirectly conjugate the cargo protein to the spore coat protein. Specifically, in some embodiments, the spore coat protein is fused to a first protein tag and the cargo protein is fused to a second protein tag that forms an isopeptide bond with the first protein tag. As is described above, the inventors have designed a conjugation method in which the spore coat protein is expressed as a fusion protein with the peptide SpyCatcher and the cargo protein is expressed as a fusion protein with the peptide SpyTag. Thus, in preferred embodiments, the first protein tag is SpyCatcher and the second protein tag is SpyTag.

Any bacterium that forms spores may be used with the methods of the present invention. Examples of such bacteria include, without limitation, Bacillus cereus, Bacillus anthracis, Bacillus thuringiensis, Bacillus megaterium, Clostridium botulinum, and Clostridium tetani. However, the anchor proteins that were identified by the inventors in Example 1 are from Bacillus subtilis. Thus, in preferred embodiments, the bacterium is from the genus Bacillus. In specific embodiments, the bacterium is Bacillus subtilis. Advantageously, B. subtilis is generally regarded as safe (GRAS) by the U.S. Food and Drug Administration (FDA). In some embodiments, the bacterium has been engineered to be germination deficient or have reduced germination efficiency through the inactivation of specific gene(s), including but not limited to: cotH, cotG, cotB, cotE, cotT, cwlD, cwlJ, gerAA, gerAB, gerAC, gerD, gerBA, gerBC, gerBB, gerKA, gerKB, gerKC, gerKD, gerE, gerM, gerQ, gerT, pdaA, pdaB, sleB, spoVAC, spoVAD, spoVAE, sscA, ypeB, and combinations thereof.

### Methods for Producing Spores:

In a third aspect, the present invention provides three methods by which the cargo-loaded spores of the present invention can be produced.

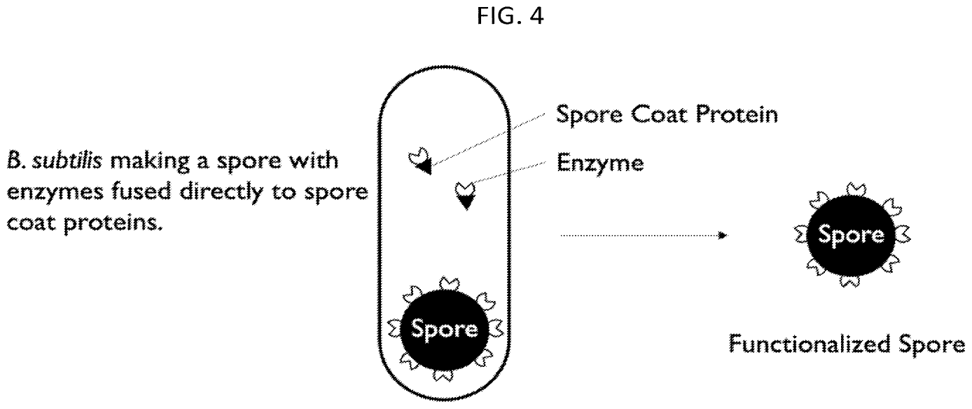

The first method for producing spores (which is depicted in FIG. 4) comprises: (a) culturing a bacterium that expresses a fusion protein comprising the spore coat protein and the cargo protein in a sporulation medium. As used herein, the term “culturing” refers to a process in which cells are grown in an artificial environment. In the present methods, the bacterium is cultured in a sporulation medium. As used herein, the term “sporulation medium” refers to any non-nutrient medium that induces spore production. Examples of sporulation medium include 2×SG media, as defined in Nicholson (Journal of bacteriology 172.1 (1990): 7-14), which is hereby incorporated by reference in its entirety. In the present methods, the bacterium should be cultured in the sporulation medium for a sufficient time to produce spores. Suitably the bacterium is cultured for 24-96 hours.

In some embodiments, this first method further comprises introducing a construct encoding the fusion protein into the bacterium prior to step (a). As used herein, the term “construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) synthetic. Constructs can be generated using conventional recombinant DNA methods. In addition to a sequence encoding the fusion protein, the construct may additionally comprise regulatory elements that facilitate transcription and translation of the fusion protein. The construct may be a vector, such as a plasmid.

As used herein, “introducing” describes a process by which exogenous polynucleotides are introduced into a recipient cell. Suitable introduction methods include, without limitation, bacteriophage or viral infection, natural transformation, electroporation, heat shock, lipofection, microinjection, and particle bombardment. In some embodiments, the constructs are introduced into the bacterium using a carrier. Suitable carriers include, but are not limited to, lipid carriers (e.g., Lipofectamine) and polymeric nanocarriers.

The second method for producing spores (which is depicted in FIG. 5) comprises: (a) culturing a bacterium that expresses (i) the spore coat protein fused to a first protein tag and (ii) the cargo protein fused to a second protein tag that specifically binds to the first protein tag in a sporulation medium. In this method, the tagged spore coat protein and tagged cargo protein are expressed independently in the same spore-producing mother cell.

In some embodiments, this second method further comprises introducing (i) a first construct encoding the spore coat protein fused to the first protein tag and (ii) a second construct encoding the cargo protein fused to the second protein tag into the bacterium prior to step (a).

In contrast, in the third method, the tagged spore coat protein and the tagged cargo protein are expressed in separate bacterial cells. Specifically, the third method for producing spores (which is depicted in FIG. 6) comprises: (a) culturing a first bacterium that expresses the spore coat protein fused to a first protein tag in a sporulation medium; (b) culturing a second bacterium that expresses a cargo protein fused to a second protein tag that specifically binds to the first protein tag in a sporulation medium; (c) isolating the cargo protein produced by the second bacterium in step (b); and (d) combining the spores produced by the first bacterium in step (a) with the cargo protein isolated from the second bacterium in step (c).

In this third method, the first bacterium that expresses the tagged spore coat protein may be any spore-producing bacterium and the second bacterium that expresses the tagged cargo protein may be any bacterium suitable for recombinant protein production (e.g., Escherichia coli, Bacillus subtilis).

In step (c), the cargo protein produced by the second bacterium may be isolated using any protein purification method known in the art (e.g., size exclusion chromatography, separation based on charge or hydrophobicity, affinity chromatography, high performance liquid chromatography, etc.). At a minimum, the second bacterium must secrete the cargo protein or it must be lysed to release the cargo protein. As used herein, the term “lysis” is used to describe disruption of a cellular membrane. Lysis causes the contents to spill out of a cell, making them accessible. Examples of lysis methods include, but are not limited to, chemical lysis, thermal lysis, mechanical lysis, and osmotic lysis. In some embodiments, the crude lysate of the second bacterium is combined with the culture of the first bacterium or with spores isolated therefrom in step (d).

In some embodiments, this third method further comprises (i) introducing a first construct encoding the spore coat protein fused to the first protein tag into the first bacterium prior to step (a), and/or (ii) introducing a second construct encoding the cargo protein fused to the second protein tag into the second bacterium prior to step (b).

In some embodiments of the second and third methods, the first protein tag forms an isopeptide bond with the second protein tag. In specific embodiments, the first protein tag is SpyCatcher and the second protein tag is a SpyTag.

Any of these three methods for producing spores may further comprise isolating spores produced by the spore-producing bacterium from the supernatant of the culture following step (a). In the third method, this this additional isolation step may be performed either before or after the tagged spores are combined with the tagged cargo protein in step (d). That is, the spores may be isolated from the supernatant of the culture of the first bacterium prior to step (d) such that isolated spores are combined with isolated cargo protein in step (d), or, alternatively, the isolated cargo protein may be added to the culture of the first bacterium in step (d) before the spores (now loaded with cargo protein) are isolated.

Following spore formation, spores are released into the environment as the mother cell undergoes programmed cell death. Thus, the spores may be easily isolated from the supernatant of the culture via centrifugation. “Centrifugation” is a method of separating molecules having different densities by spinning them in solution around an axis at high speed. Examples of suitable centrifugation conditions for isolating bacterial spores from cell culture include 2 minutes at 21,000 g or 5 minutes at 3,000 g.

While any of these methods can be used to produce the spores described herein, we note that the second and third method may be preferable because fusion of the cargo protein and spore coat protein to short protein tags (e.g., the 14 amino acid SpyTag) greatly reduces the chance of these proteins misfolding as compared to when they are expressed as a single cargo protein-spore coat protein fusion protein.

### Pharmaceutical Compositions:

In a fourth aspect, the present invention provides pharmaceutical compositions comprising a spore described herein and a pharmaceutically acceptable carrier.

Pharmaceutically acceptable carriers are known in the art and include, but are not limited to, diluents (e.g., Tris-HCl, acetate, phosphate), preservatives (e.g., thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. Examples of nonaqueous solvents are propylene glycol, polyethylene glycol, vegetable oils (e.g., olive oil), and injectable organic esters (e.g., ethyl oleate). Aqueous carriers include isotonic solutions, alcoholic/aqueous solutions, emulsions, and suspensions, including saline and buffered media.

The pharmaceutical compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol).

### Methods for Delivering a Cargo Protein to a Subject:

In a fifth aspect, the present invention provides methods for delivering a cargo protein to a subject in need thereof. The methods comprise administering a spore or pharmaceutical composition described herein to the subject.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injections such as intravenous administration, intra-arterial administration, intramuscular administration, intradermal administration, intrathecal administration, and subcutaneous administration. Administration can be continuous or intermittent. In certain embodiments, the composition is administered orally.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

In some embodiments, the cargo protein is delivered to a specific cell-type or tissue within the subject. For example, in some embodiments, the cargo protein is delivered to the intestinal tract of the subject.

Antibiotics are prescribed roughly 275 million times per year in the United States. However, some antibiotics are non-specific, i.e., they kill the pathogenic microbes causing the infection, but they also commonly kill many of the beneficial bacteria that live in our intestinal tract. Death of these beneficial bacteria can cause severe problems. For example, antibiotic-induced collapse of the native microbiome creates opportunities for pathogenic bacteria such as Clostridioides difficile to colonize the gut. Annually, there are about 450,000 infections and 30,000 deaths caused by C. difficile in the United States alone. C. difficile infection is the most common hospital-acquired infection, and it was estimated in 2009 to have been responsible for more than 2% of all hospital costs in the United States. Additionally, antibiotic-induced imbalances in the gut microbiome (i.e., dysbiosis) are associated with negative health outcomes including depression, certain cancers, colitis, and other chronic diseases. Thus, in some embodiments, the methods of the present invention are used to deliver an enzyme that degrades antibiotics to the intestinal tract to prevent antibiotic-induced collapse of the microbiome. In these embodiments, the spore composition must be formulated such that it is capable of reaching the intestinal tract of the subject. For example, the composition may be formulated as an orally administered liquid or pill, as oral administration of spore-displayed enzymes should allow them to pass through the stomach and into the intestines where they can degrade antibiotics.

Proteins are fragile, and efficiently delivering functional proteins to the intestinal tract can be challenging because they are destabilized and degraded in the stomach and intestines by low pH, varying salt concentrations, and proteinases. For this reason, enzymes are often delivered to the gut within the cells in which they are produced (e.g., SYNB 1618 from Synlogic). However, such products contain genetic material encoding the enzymes and are therefore not agenetic. Because spores stabilize proteins and can be made to be agenetic via trans-loading, they could be used to deliver antibiotic-degrading enzymes while imposing no risk of spreading antibiotic resistance genes. Thus, the agenetic spores of the present invention offer a safer means to deliver enzymes to the intestinal tract. Delivery of antibiotic-degrading enzymes is one of many potential applications of the engineered spores described herein.

It should be apparent to those skilled in the art that many additional modifications besides those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### EXAMPLES

### Example 1: Identification of Novel Bacillus subtilis Spore Coat Anchor Proteins

To identify novel Bacillus subtilis spore coat anchor proteins, we first performed a thorough literature search to compile a list of all known B. subtilis spore coat proteins. Of these, some had already been shown to be functional enzyme anchor proteins while others had been shown to be non-functional. We created a final list of 44 spore coat proteins and designed 88 constructs in which we modified the B. subtilis genome to encode fusion proteins in which the enzyme beta-glucosidase (GusA) is fused to the N- or C-terminus of each spore coat protein (SCP). We were able to synthesize 52 of the 88 designed constructs.

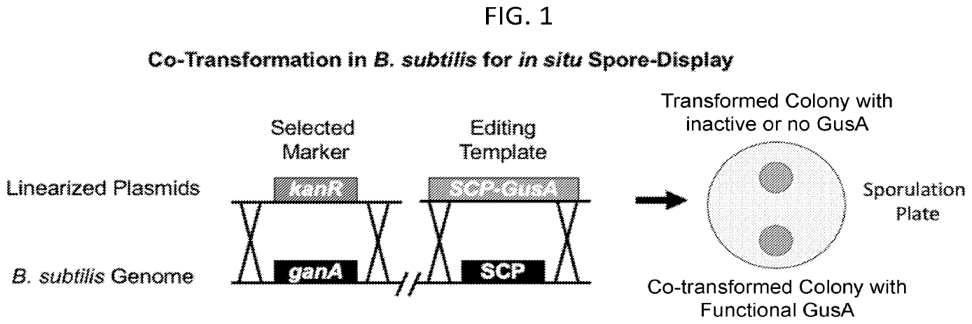

To form each of these constructs, we used a co-transformation method that enabled scarless editing of the SCP loci (FIG. 1). In this method, wild-type B. subtilis strain 168 is co-transformed with two DNA molecules: one molecule carrying a kanamycin resistance (kanR) gene that is integrated at the unessential ganA locus and a second molecule carrying the sequence encoding the SCP-GusA fusion protein that is integrated at the SCP locus. The co-transformed cells are plated on agar plates containing 2×SG sporulation medium, kanamycin, and the chromogenic substrate X-gluc. As a result, only cells that took up the kanR gene can grow on the plates. A percentage of the population (~5%) took up both DNA molecules. If the sequence encoding the SCP-GusA fusion protein was also integrated into the genome, then, as the cells sporulated, the SCP-GusA fusion protein would be expressed. Successful integration, expression, and folding of the SCP-GusA fusion protein would then result in the loading of functional GusA enzyme onto the spores produced by the bacteria. Functional GusA would then react with the X-Gluc in the substrate and turn the colony blue. Thus, the formation of blue colonies in this assay indicates that a spore coat protein forms a functional construct that could be loaded to the spore surface. Colony-PCR was used to confirm genomic integration of the SCP-GusA construct at each native locus.

Based on this screen, 34 of the 52 synthesized constructs yielded functional fusion proteins (Table 1). The 34 successful constructs represent 26 unique anchor proteins (Table 2), 13 of which are novel enzyme anchor proteins (Table 3).

TABLE 1
List of the 34 constructs that produced functional fusion proteins
CgeA-C-GusA-His
CgeA-N-GusA-His
CotA-C-GusA-His
CotB-C-GusA-His
CotC-C-GusA-His
CotC-N-GusA-His
CotG-C-GusA-His
CotG-N-GusA-His
CotI-C-GusA-His
CotI-N-GusA-His
CotJA-C-GusA-His
CotM-C-GusA-His
CotS-C-GusA-His
CotU-C-GusA-His
CotV-C-GusA-His
CotW-C-GusA-His
CotW-N-GusA-His
CotX-C-GusA-His
CotY-C-GusA-His
CotY-N-GusA-His
EcsB-C-GusA-His
OxdD-C-GusA-His
OxdD-N-GusA-His
PdaB-C-GusA-His
PdaB-N-GusA-His
PrtG-C-GusA-His
SpaD-C-GusA-His
SscA-C-GusA-His
YhaX-C-GusA-His
YhjR-N-GusA-His
YqfQ-C-GusA-His
YrkC-C-GusA-His
YtcC-C-GusA-His
YtrH-N-GusA-His

TABLE 2
List of the 26 functional anchor proteins

| Anchor protein | SEQ ID NO: |
|---|---|
| CgeA | 1 |
| CotA | 2 |
| CotB | 3 |
| CotC | 4 |
| CotG | 5 |
| CotI | 6 |
| CotJA | 7 |
| CotM | 8 |
| CotS | 9 |
| CotU | 10 |
| CotV | 11 |
| CotW | 12 |
| CotX | 13 |
| CotY | 14 |
| EcsB | 15 |
| OxdD | 16 |
| PdaB | 17 |
| PrtG | 18 |
| SpaD | 19 |
| SscA | 20 |
| YhaX | 21 |
| YhjR | 22 |
| YqfQ | 23 |
| YrkC | 24 |
| YtcC | 25 |
| YtrH | 26 |

TABLE 3
List of the 13 novel functional anchor proteins
and gene sequences encoding them

| Anchor protein | SEQ ID NO: |
|---|---|
| CotI | 6 |
| CotU | 10 |
| EcsB | 15 |
| PdaB | 17 |
| PrtG | 18 |
| SpaD | 19 |
| SscA | 20 |
| YhaX | 21 |
| YhjR | 22 |
| YqfQ | 23 |
| YrkcC | 24 |
| YtcC | 25 |
| YtrH | 26 |

### Example 2: Assessment of Spore-Displayed Cargo Protein Activity and Accessibility

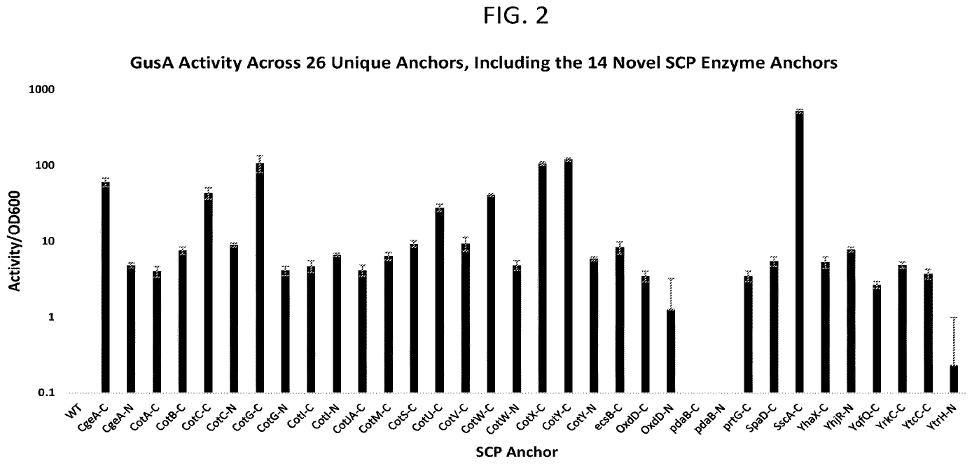

We tested the activity of the SCP-GusA fusion proteins identified in Example 1. Specifically, using a Gus activity assay, we tested the activity of the 34 constructs listed in Table 1, which encode the enzyme GusA with its N- or C-terminus fused to the 26 SCP anchors listed in Table 2. The activity of spore-displayed GusA was found to vary by spore coat anchor protein, and the highest GusA activity was seen with the anchor protein SscA-C(FIG. 2).

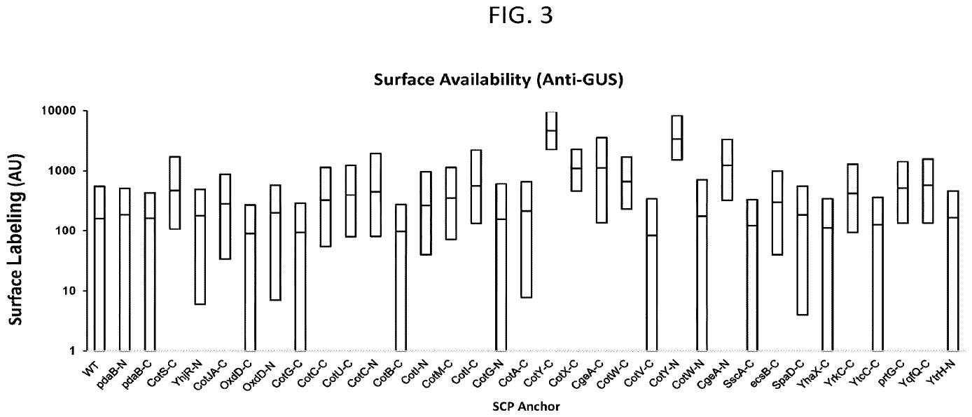

Additionally, we tested the surface availability of GusA expressed from the 34 different constructs by labeling recombinant spores with an anti-GusA antibody. Purified spores were incubated with a primary anti-GusA polyclonal antibody and a secondary fluorescent antibody and were analyzed using flow cytometry. The surface accessibility of spore-displayed GusA was found to vary widely by spore coat anchor protein (FIG. 3).

### Example 3: Assembly of Cargo-Loaded Spores

In the following example, we describe three methods by which spores comprising a cargo protein fused to a spore coat anchor protein can be produced.

### 1) Cis-Loading Via Direct Fusion

Assembly of cis-loaded spores by direct fusion of the cargo protein to a spore coat anchor protein is depicted in FIG. 4. Briefly, B. subtilis strains containing the DNA encoding the desired SCP-cargo fusion protein are cultured in a starvation media that induces sporulation. After 48-60 hours, most of the cell population will have sporulated. Upon complete sporulation, the functionalized spores can be easily purified by centrifugation.

### 2) Cis-Loading Using the SpyCatcher-SpyTag System

Assembly of cis-loaded spores using the SpyCatcher-SpyTag system is depicted in FIG. 5. SpyCatcher is fused to the desired spore coat protein. The cargo protein is expressed in the same mother cell fused to a SpyTag. The mother cell is cultured in starvation sporulation media. As the spore forms in the mother cell, the SpyCatcher and SpyTag spontaneously form a covalent isopeptide bond, conjugating the spore coat protein to the cargo protein. Upon complete sporulation, the functionalized spores can be easily purified by centrifugation.

### 3) Trans-Loading Using the SpyCatcher-SpyTag System

Assembly of trans-loaded spores using the SpyCatcher-SpyTag system is depicted in FIG. 6. SpyCatcher is fused to the desired spore coat protein. Following complete sporulation, SpyCatcher-functionalized spores can be purified by centrifugation. The cargo protein-SpyTag fusion protein is produced in a second production host. Following expression, the cargo protein-SpyTag fusion protein is released from the production host through lysis and purified if desired. The purified spores and cargo protein can then be mixed together in a buffer solution (e.g., PBS, pH=7.0) to load the cargo protein onto the surface of the spores. After assembly, the functionalized spores can be easily purified by centrifugation.

### Example 4: Assessment of Cargo Protein Stability

Enzymes are biological catalysts that are used in chemical manufacturing, agriculture, bioremediation, therapeutics, and other industries. Because spore display of proteins often confers increased stability to the displayed protein, this technology has potential uses in all these industries. We predict that the stabilization conferred by spore display will allow enzyme cargo proteins to retain their activity in the face of various stability challenges including time, temperature, pH, proteinases, solvents, detergents, and more. Thus, we are testing the ability of the B. subtilis anchor proteins identified in Example 1 to stabilize conjugated enzymes against all these stability challenges. Our preliminary data is presented below.

### Temperature Challenge

Heat can help a chemical reaction move faster, and many enzymes can catalyze reactions more quickly at higher temperatures. But if the temperature is too high, an enzyme will become destabilized and/or denatured and will be unable to catalyze the reaction. Thus, stabilizing enzymes against temperature challenges allows reactions to occur faster and longer at higher temperatures. Many technologies use immobilization to enhance enzyme stability.

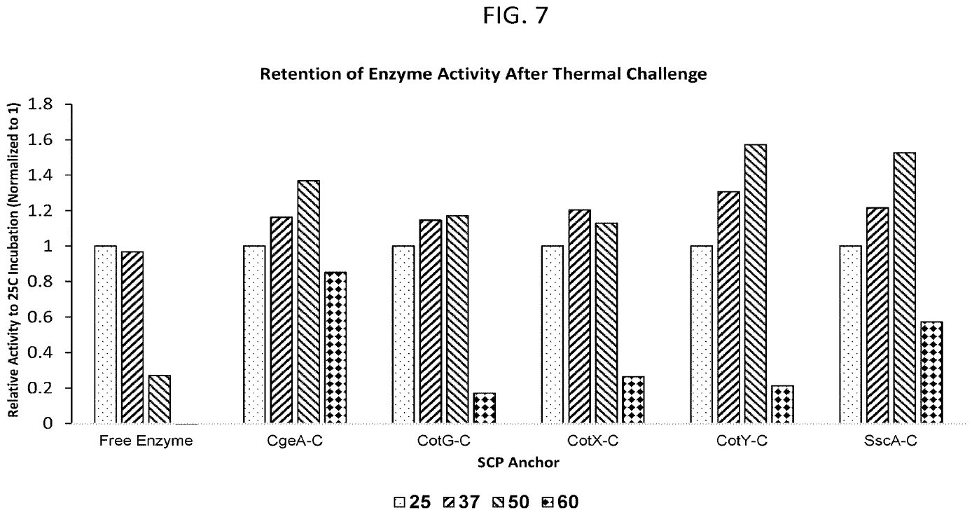

We tested whether fusing the enzyme GusA to various B. subtilis anchor proteins identified in Example 1 would stabilize it against temperature challenge. Specifically, GusA was fused to the spore coat proteins CgeA-C, CotG-C, CotX-C, CotY-C, and SscA-C and spore-displayed GusA was incubated at 25, 37, 50, and 60° C. for a stability challenge. Loss of free GusA enzyme activity begins at 37° C. All tested spore-displayed enzymes retained or gained activity up to 50° C., but the magnitude of the effect varies with anchor protein (FIG. 7). For example, while free GusA enzyme completely loses activity after incubation at 60° C., GusA fused to CgeA-C or SscA-C retains>50% of activity at this temperature. pH Challenge

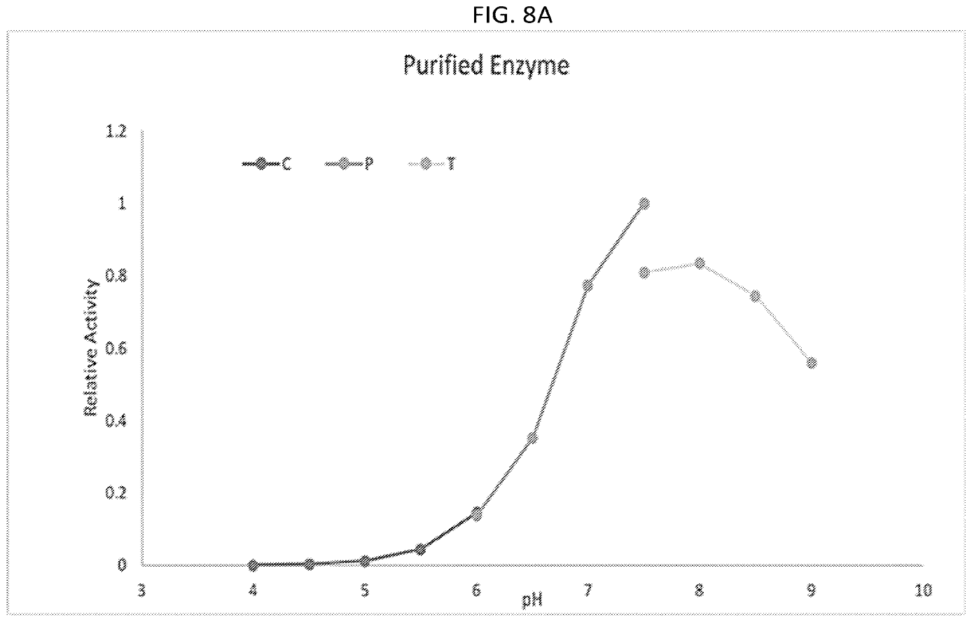

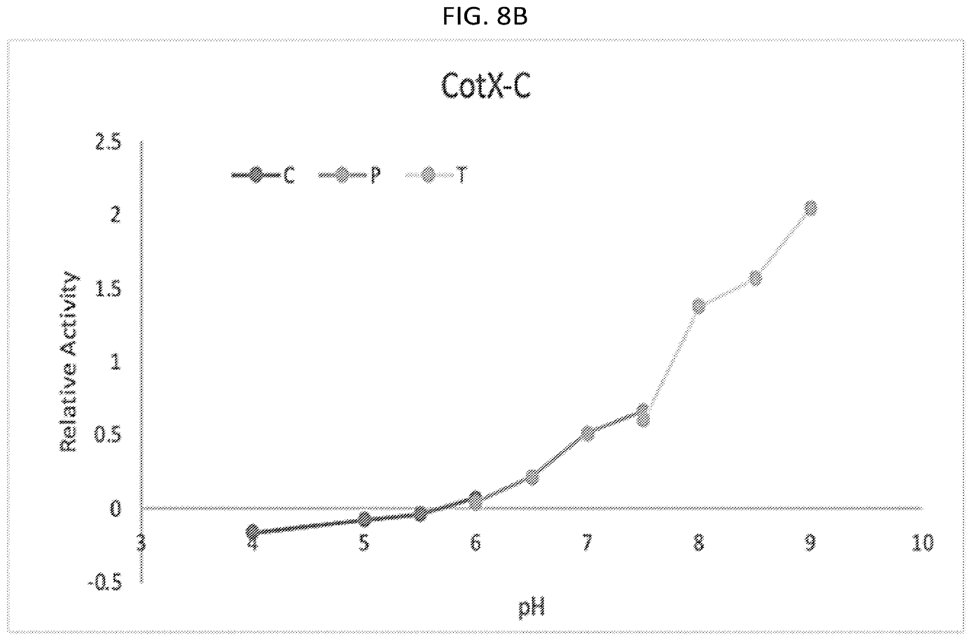

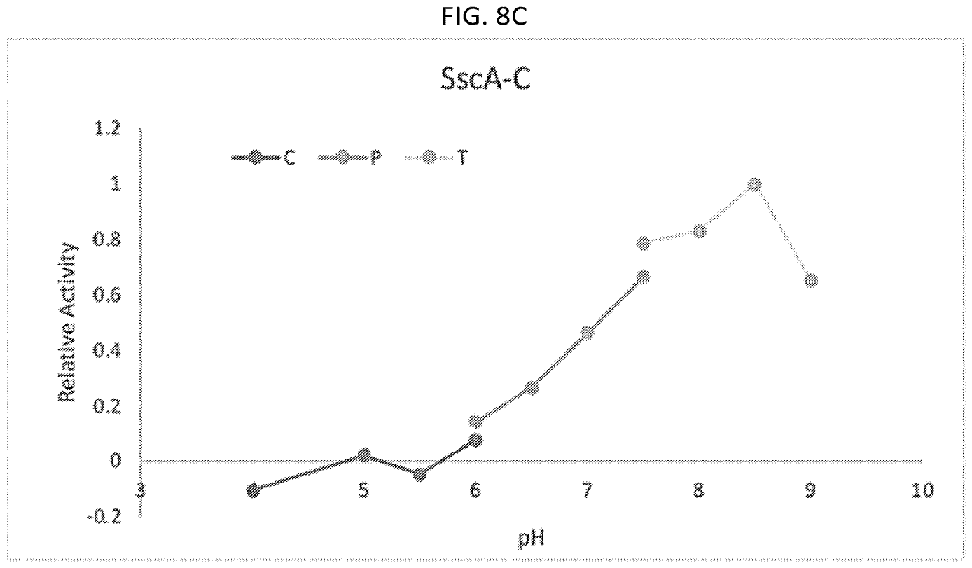

Spore display may also be used to increase enzyme stability against pH extremes. We have tested the ability of the anchor proteins CotX-C(FIG. 8B) and SscA-C(FIG. 8C) to confer stability against extreme pH as compared to free enzyme (FIG. 8A), and we found that conjugation of the enzyme GusA to these spore coat proteins results in higher relative activity than free enzyme at pH>8.

### Example 5: Assessment of a Different Cargo Protein

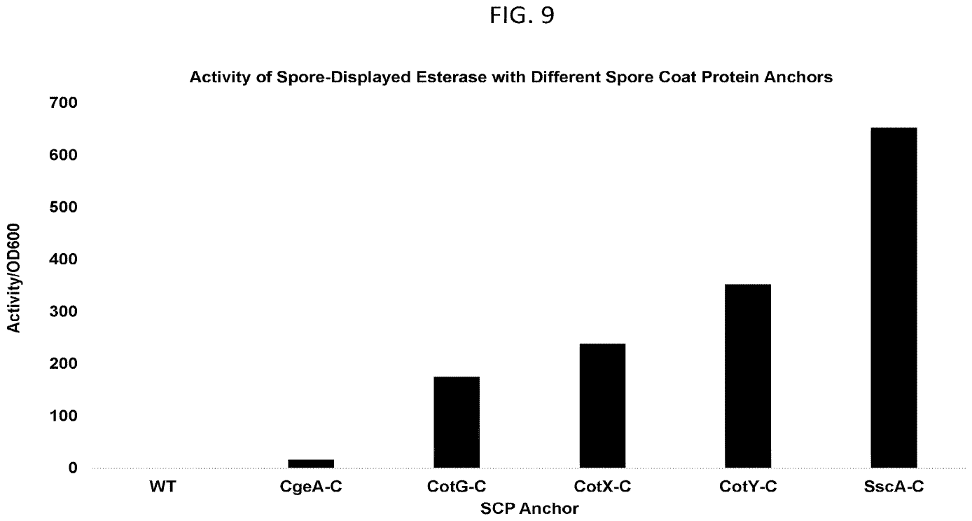

We also tested the activity of a second enzyme: DuraPETase, an engineered variant of the esterase from Ideonella sakaiensis (ACS Catalysis 11(3): 1340-1350, 2021). DuraPETase was fused to the SCP anchors CgeA-C, CotG-C, CotX-C, CotY-C, and SscA-C using a para-nitrophenyl acetate esterase activity assay (Sci Total Environ 709:136138, 2020). As with GusA, the activity of spore-displayed DuraPETase was found to vary by spore coat anchor protein, and the highest GusA activity was seen with the anchor protein SscA (FIG. 9). In fact, the esterase activity with SscA was greater than any previously tested SCP.

What is claimed:

1. A bacterial spore comprising a cargo protein conjugated to a spore coat protein.

2. The spore of claim 1, wherein the spore coat protein is selected from the proteins listed in Table 2.

3. The spore of claim 1, wherein the spore coat protein is selected from the proteins listed in Table 3.

4. The spore of any one of claims 1-3, wherein the spore coat protein is fused to a first protein tag, and wherein the cargo protein is fused to a second protein tag that specifically binds to the first protein tag.

5. The spore of claim 4, wherein the second protein tag forms an isopeptide bond with the first protein tag.

6. A bacterium that produces the spore of any one of claims 1-5.

7. The bacterium of claim 6, wherein the bacterium expresses the spore coat protein and the cargo protein as a fusion protein.

8. The bacterium of claim 6, wherein the bacterium expresses the spore coat protein and the cargo protein as individual proteins.

9. The bacterium of claim 8, wherein the spore coat protein is fused to a first protein tag, and wherein the cargo protein is fused to a second protein tag that specifically binds to the first protein tag.

10. The bacterium of claim 9, wherein the second protein tag forms an isopeptide bond with the first protein tag.

11. The bacterium of any one of claims 6-10, wherein the bacterium is from the genus Bacillus.

12. The bacterium of claim 11, wherein the bacterium is Bacillus subtilis.

13. A method for producing the spore of any one of claims 1-3, the method comprising:
a) culturing a bacterium in a sporulation medium, wherein the bacterium expresses a fusion protein comprising the spore coat protein and the cargo protein.

14. A method for producing the spore of any one of claims 1-5, the method comprising:
a) culturing a bacterium in a sporulation medium, wherein the bacterium expresses (i) the spore coat protein fused to a first protein tag and (ii) the cargo protein fused to a second protein tag that specifically binds to the first protein tag.

15. The method of claim 13 or 14 further comprising:
b) isolating spores produced by the bacterium from the supernatant of the culture.

16. The method of claim 15, wherein the spores are isolated via centrifugation.

17. A method for producing the spore of any one of claims 1-5, the method comprising:
a) culturing a first bacterium in a sporulation medium, wherein the first bacterium expresses the spore coat protein fused to a first protein tag;
b) culturing a second bacterium in a sporulation medium, wherein the second bacterium expresses the cargo protein fused to a second protein tag that specifically binds to the first protein tag;
c) isolating the cargo protein produced by the second bacterium in step (b);
d) combining with the spores produced by the first bacterium in step (a) with the cargo protein isolated from the second bacterium in step (c).

18. The method of claim 17 further comprising isolating spores produced by the first bacterium in step (a) from the supernatant of the culture prior to step (d).

19. The method of claim 18, wherein the spores are isolated via centrifugation.

20. The method of claim 14 or 17, wherein the second protein tag forms an isopeptide bond with the first protein tag.

21. A spore produced by the method of any one of claims 13-20.

22. The spore of any one of claim 1-5 or 21, wherein the cargo protein is an enzyme.

23. A pharmaceutical composition comprising a pharmaceutically acceptable carrier and the spore of any one of claims 1-5, 21, and 22.

24. A method for delivering a cargo protein to a subject in need thereof, the method comprising administering the spore of any one of claims 1-5, 21, and 22 or the composition of claim 23 to the subject.

25. The method of claim 24, wherein the cargo protein is an enzyme.

26. The method of claim 25, wherein the enzyme degrades antibiotics.

27. The method of any one of claims 24-26, wherein the cargo protein is delivered to the intestinal tract of the subject.

28. The method of any one of claims 24-27, wherein the composition is administered orally.
